(19) United States
(12) Patent Application Publication
(10) Pub. No.: US 2021/0236416 A1
(43) Pub. Date: Aug. 5, 2021

(54) TREATMENT OF SKIN DISORDERS WITH TOPICAL ROFLUMILAST COMBINATION COMPOSITIONS

(71) Applicant: Sol-Gel Technologies Ltd., Ness Ziona (IL)

(72) Inventors: Moshe ARKIN, Kfar Shmaryahu (IL); Marcel ZIGHELBOIM, Kiryat Motzkin (IL); Karine NEIMANN, Ness Ziona (IL)

(73) Assignee: Sol-Gel Technologies Ltd., Ness Ziona (IL)

(21) Appl. No.: 17/166,164

(22) Filed: Feb. 3, 2021

Related U.S. Application Data
(60) Provisional application No. 62/969,206, filed on Feb. 3, 2020.

(51) Int. Cl. A61K 9/00; A61K 31/4436; A61K 31/327; A61K 9/06; A61K 9/48

(52) U.S. Cl. CPC A61K 9/0014; A61K 31/4436; A61K 31/327; A61K 9/06; A61K 9/4891; A61K 9/4858; A61K 9/4866; A61K 9/4875; A61K 9/4833; A61K 9/485

(57) ABSTRACT

Provided herein is a topical combination composition comprising roflumilast, roflumilast N-oxide or pharmaceutically acceptable salt thereof, and at least one additional active agent selected from a retinoid, benzoyl peroxide (BPO) and combinations thereof. The active agents in the composition of this invention are in encapsulated or non-encapsulated form, according to need.

The above composition is useful for the treatment, prevention or alleviation of a skin disorder selected from acne and rosacea and exhibits synergistic and/or additive effects allowing to reduce the active agents amounts in the topical combination compositions.

### CROSS REFERENCE TO RELATED APPLICATIONS

This application claims the benefit of U.S. Provisional Application No. 62/969,206, filed on Feb. 3, 2020 (and entitled Treatment of Skin Disorders with Topical Roflumilast Combination Compositions), which is incorporated in its entirety herein by reference.

### FIELD OF THE INVENTION

The present disclosure, in some embodiments, relates to a method of treatment of a skin disorder by topical administration to a subject in need thereof a combination composition comprising roflumilast and at least one additional active agent selected from a retinoid and benzoyl peroxide (BPO). The compositions of this invention are useful for the treatment, prevention or amelioration of skin disorders selected from acne and rosacea.

### BACKGROUND

Skin disorders (also known as skin conditions) in general and acne/rosacea in particular vary greatly in symptoms and in severity. They are commonly treated with systemic and/or topical medicaments. Topical medicaments, while not always available, have the advantage of avoiding systemic side-effects. On the other hand, also topical skin disorder treatments are sometimes accompanied by undesirable side-effects, especially at high doses.

### SUMMARY OF THE INVENTION

This invention provides a topical combination composition comprising roflumilast, roflumilast N-oxide, or pharmaceutically acceptable salt thereof and at least one additional active agent selected from a retinoid, benzoyl peroxide (BPO) and combinations thereof. The active agents in the composition of this invention are in encapsulated or non-encapsulated form, according to need.

The above compositions are useful for the treatment, prevention or alleviation of a skin disorder selected from, acne and rosacea, and exhibit synergistic and/or additive effects allowing to reduce the active agents amounts in the combination compositions.

The addition of the at least one additional active agent to roflumilast potentiates the roflumilast therapeutic effect. The present disclosure provides novel combination compositions of roflumilast with at least one additional active agent selected from a retinoid, BPO and combinations thereof and aims at minimizing undesirable side-effects, while using reduced active agents amounts.

There is an unmet need for methods of treatment of a skin disorder using topical combination compositions comprising roflumilast, roflumilast N-oxide, or pharmaceutically acceptable salt thereof and at least one additional active agent selected from a retinoid, BPO and combinations thereof in lower doses than the marketed single drug products, devoid of serious side-effects, which may be used for extended periods of time.

### DETAILED DESCRIPTION OF THE INVENTION

The present invention provides topical combination compositions comprising roflumilast, roflumilast N-oxide, or pharmaceutically acceptable salt thereof and at least one additional active agent selected from at least one retinoid, benzoyl peroxide (BPO) and combinations thereof, and methods of treatment useful for the treatment, prevention and amelioration of a skin disorder selected from acne and rosacea.

The at least one retinoid in the combination compositions of this disclosure is selected from adapalene, tretinoin, trifarotene, tazarotene and combinations thereof.

It occurred to the present inventor, that topical combination compositions comprising roflumilast and at least one additional active agent selected from at least one retinoid, benzoyl peroxide (BPO) and combinations thereof may allow treatment of skin disorders for longer periods of time, exhibit improved therapeutic effects and also exhibit synergistic or additive effects in the treatment of a skin disorder. As a result, the combination compositions of this disclosure allow using lower dosage of the actives and thus diminish the product's side-effects.

In some embodiments, there is provided a topical composition comprising from about 0.25% w/w to about 3.0% w/w roflumilast, roflumilast N-oxide, or pharmaceutically acceptable salt thereof, from about 0.001% w/w to about 0.5% w/w of at least one retinoid selected from tretinoin, trifarotene and adapalene, and a carrier suitable for topical administration.

In some embodiments, there is provided a topical composition comprising from about 0.25% w/w to about 3.0% w/w roflumilast, roflumilast N-oxide, or pharmaceutically acceptable salt thereof and from about 2% w/w to about 10% w/w benzoyl peroxide (BPO) and combinations thereof, and a carrier suitable for topical administration.

In some embodiments, there is provided a topical composition comprising from about 0.25% w/w to about 3.0% w/w roflumilast, roflumilast N-oxide, or pharmaceutically acceptable salt thereof, from about 0.001% w/w to about 0.5% w/w of at least one retinoid selected from tretinoin, trifarotene and adapalene, from about 2% w/w to about 10% w/w benzoyl peroxide (BPO) and combinations thereof, and a carrier suitable for topical administration.

In some embodiments, there is provided a topical composition comprising from about 0.25% w/w to about 3.0% w/w roflumilast, roflumilast N-oxide, or pharmaceutically acceptable salt thereof, from about 0.001% w/w to about 0.5% w/w tazarotene, and a carrier suitable for topical administration, wherein the carrier suitable for topical administration does not comprise hexylene glycol or diethylene glycol monoethyl ether.

In some embodiments, there is provided a topical composition comprising from about 0.25% w/w to about 3.0% w/w roflumilast, roflumilast N-oxide, or pharmaceutically acceptable salt thereof, from about 0.001% w/w to about 0.5% w/w tazarotene, from about 2% w/w to about 10% w/w benzoyl peroxide (BPO) and combinations thereof, and a carrier suitable for topical administration, wherein the carrier suitable for topical administration does not comprise hexylene glycol or diethylene glycol monoethyl ether.

In some embodiments, there is provided a composition comprising roflumilast, roflumilast N-oxide, or pharmaceutically acceptable salt thereof and at least one additional active agent selected from at least one retinoid and benzoyl peroxide, wherein the active agents are fully dissolved or partly dissolved and partly suspended in a carrier comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof (see Examples 1-5).

In some embodiments, there is provided a composition comprising roflumilast and at least one additional active agent selected from at least one retinoid and benzoyl peroxide, wherein at least one of all the active agents is encapsulated.

In some embodiments, there is provided a composition comprising roflumilast, roflumilast N-oxide, or pharmaceutically acceptable salt thereof and at least one additional active agent selected from at least one retinoid and benzoyl peroxide, wherein all the active agents of the composition are encapsulated (see Examples 6-11, silicone dioxide (SiO₂) encapsulated active agents).

The advantages of the active agents' encapsulation are on the one hand the improved chemical stability of the active agents in the composition and on the other hand its improved therapeutic effect, including reduced side-effects and sustained-release effect.

The roflumilast combination composition of this invention has a double advantage vs. the use of roflumilast, a retinoid or BPO as single drugs: on the one hand the roflumilast has the potential to alleviate the retinoid or BPO side-effects, especially at high concentrations, and on the other hand the synergistic and/or additive effect may enable using lower active agent amounts. The addition of a retinoid and/or BPO to roflumilast potentiates the roflumilast therapeutic effect.

Active Agents in the Compositions of this Disclosure:

### Roflumilast (a PDE4 Inhibitor)

Phosphodiesterase Type 4 inhibitors are blocking the degradation of cyclic adenosine monophosphate (cAMP) by phosphodiesterase 4 (PDE4). Some of the known PDE4 inhibitors are roflumilast, apremilast, crisaborole, rolipram. cilomilast, ibudilast and piclamilast.

Roflumilast was the first FDA-approved PDE4 inhibitor. This PDE4 inhibitor is FDA-approved (Daliresp®) and EMA-approved (Daxas®) for oral treatment of severe chronic obstructive pulmonary disease (COPD).

The systemic administration of roflumilast is accompanied by side-effects including inter alia emesis and diarrhea, which is a big drawback to the oral administration. The topical administration of roflumilast comprising combination drugs provided in this disclosure avoids the systemic side-effects.

Roflumilast has a very low calculated water solubility (0.0062 mg/mL, according to the roflumilast DrugBank monograph), which is a problem for a topical product.

The following routes for improving the roflumilast solubility and skin permeability are put forward in this disclosure:
a. Use of potent solvents, like DMSO, propylene glycol, a polyethylene glycol (PEG), ethanol, isopropyl alcohol, dimethyl isosorbide, isopropyl myristate, oleic acid, glycofurol and combinations thereof (see Examples 1-5).
b. Use of finely milled or micronized roflumilast.
c. Use a soluble roflumilast salt as active agent.
d. Use of a soluble roflumilast pro-drug.
e. Use of a roflumilast-cyclodextrin complex.
f. Use of an amorphous form of roflumilast.
g. Use of a roflumilast solid dispersion.

The term “Roflumilast” as used herein includes roflumilast free base, its pharmacologically active metabolites including roflumilast N-oxide, or its pharmaceutically acceptable salt. Preferably, roflumilast is roflumilast free base. Roflumilast present in the composition according to present invention can be in crystalline form or amorphous form.

Suitable salts for roflumilast include inorganic and organic acids. In other embodiments, non-limited examples of acids include: hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in salt preparation of roflumilast

### Retinoid

Retinoids are a class of chemical compounds structurally related to Vitamin A which are effective in the treatment of a number of skin disorders, like acne and psoriasis.

Long-term high intake of retinoids causes a number of side-effects.

The retinoids belong to several generations the main members of which are tretinoin, trifarotene, adapalene and tazarotene.

Tretinoin, a first-generation retinoid (also known as all-trans retinoic acid or ATRA) is used topically for the treatment of acne.

Adapalene, a third-generation retinoid, is used topically for the treatment of mild to moderate acne.

Tazarotene (marketed as Tazorac, Avage, Zorac and Fabior) is a third-generation prescription topical retinoid sold as a cream, gel, or foam. Tazarotene exhibits side-effects like itching, redness, burning and stinging, especially on long-term treatment.

Trifarotene is a first-in-class, fourth-generation topical retinoid, approved by FDA in 2019 as Aklief 0.005% cream for the topical treatment of acne vulgaris.

### Benzoyl Peroxide (BPO)

BPO topical gel (alone or in combination with another active agent selected from adapalene, clindamycin, erythromycin) is used in the topical treatment of acne and based on recent phase 3 results BPO was found effective for treating rosacea.

Topical BPO treatment is accompanied by side-effects like skin irritation, itching, peeling and reddened skin. The encapsulated-BPO compositions of the instant disclosure reduce these BPO side-effects. BPO in the examples of this disclosure is encapsulated in silicon dioxide (SiO₂) according to Sol-Gel's technology (see Examples 6-11, U.S. Pat. No. 9,687,465 and published U.S. Patent Application No. 2018147165 (to Sol-Gel Technologies), whose contents are enclosed herein in their entirety).

Due to its peroxide chemical structure, BPO presents several problems:

BPO is a strong oxidant, which may compromise the chemical stability of the other active agents in the combination compositions of this invention; and

BPO has side-effects like skin irritation, itching, peeling and reddened skin.

The BPO-comprising compositions of this invention use finely milled or micronized BPO as raw-material and several solutions to the above side-effects:

BPO encapsulation according to Examples 6-11, U.S. Pat. No. 9,687,465 and published U.S. Patent Application No. 2018147165 (to Sol-Gel Technologies), whose contents are enclosed herein in their entirety, thus protecting the other active agents in a BPO-combination composition from the BPO oxidation effect and minimizing the BPO skin irritation side-effect.

Skin Disorders Treated with the Compositions of this Disclosure

The skin disorders treated with the compositions of this disclosure are selected from acne and rosacea. The acne is selected from acne vulgaris, papulopustular acne and nodular acne, and the rosacea is selected from erythematotelangiectatic rosacea, papulopustular rosacea, rhinophyma rosacea and ocular rosacea.

### Acne

Acne is a chronic inflammatory disease of the pilosebaceous unit resulting from androgen-induced increased sebum production, altered keratinization, inflammation, and bacterial colonisation of hair follicles on the face, neck, chest, and back by Propionibacterium acnes (P. acnes). Although early colonisation with P. acnes and family history might have important roles in the disease, exactly what triggers acne and how treatment affects the course of the disease remains unclear (Williams H. C. et al., The Lancet, Vol. 379. January 2012, pp. 361-372).

There is no ideal treatment for acne. Good quality evidence on comparative effectiveness of common topical and systemic acne therapies is scarce. Topical therapies including benzoyl peroxide, retinoids, and antibiotics when used in combination usually improve control of mild to moderate acne, but suffer from side-effects. Treatment with combined oral contraceptives can help women with acne. Patients with more severe inflammatory acne usually need oral antibiotics combined with topical benzoyl peroxide to decrease antibiotic-resistant organisms.

Oral isotretinoin is the most effective therapy and is used early in severe disease, but its use is limited by teratogenicity and other side-effects.

The treatment of acne with the composition of this invention allows using lower amounts of active agents and therefore results in reduced side-effects, due to the additive and/or synergistic effects.

### Rosacea (Acne Rosacea)

Rosacea is a chronic skin disease that affects more than 16 million Americans. The cause of rosacea is still unknown. However, research has allowed doctors to find ways to treat the condition by minimizing its symptoms (Cynthia Cobb, Healthline May 21, 2018).

There are four subtypes of rosacea. Each subtype has its own set of symptoms. It is possible to have more than one subtype of rosacea at a time.

Rosacea's typical symptom is small, red, pus-filled bumps on the skin that are present during flare-ups. Typically, rosacea affects only skin on the nose, cheeks, and forehead.

Flare-ups often occur in cycles. This means that one will experience symptoms for weeks or months at a time, the symptoms will go away, and then return.

The four types of rosacea are:

Subtype one, known as erythematotelangiectatic rosaca (ER), is associated with facial redness, flushing, and visible blood vessels.

Subtype two, papulopustular (or acne) rosacea, is associated with acne-like breakouts, and often affects middle-aged women.

Subtype three, known as rhinophyma, is a rare form associated with thickening of the skin on your nose. It usually affects men and is often accompanied by another subtype of rosacea.

Subtype four is known as ocular rosacea, and its symptoms are centered on the eye area.

The present invention provides topical combination compositions comprising roflumilast, roflumilast N-oxide, or pharmaceutically acceptable salt thereof and at least one additional active agent selected from at least one retinoid, benzoyl peroxide (BPO) and combinations thereof. Table 1 provides exemplary combinations useful for the treatment, prevention or alleviation of a skin disorder selected from acne and rosacea.

TABLE 1
Exemplary Combinations of Active Agents
and Classes

| | | | |
|---|---|---|---|
| Act. Agent Class/Comb. | ROFL | RET | BPO |

Two-components roflumilast combinations

| | | | |
|---|---|---|---|
| ROFL/ADAP | + | + | |
| ROFL/TRET | + | + | |
| ROFL/TRIF | + | + | |
| ROFL/TAZA | + | + | |
| ROFL/BPO | + | | + |

Three-components roflumilast combinations

| | | | |
|---|---|---|---|
| ROFL/ADAP/BPO | + | + | + |
| ROFL/TRET/BPO | + | + | + |
| ROFL/TRIF/BPO | + | + | + |
| ROFL/TAZA/BPO | + | + | + |

Legend:
Roflumilast (ROFL), Retinoid (RET), Benzoyl peroxide (BPO), Adapalene (ADAP), Tretinoin (TRET), Trifarotene (TRIF), Tazarotene (TAZA).

The frequency of administration of the composition of this invention can be determined empirically. Exemplary frequencies are once daily, twice daily, weekly, bi-weekly and monthly. Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

Pharmaceutical carriers or vehicles suitable for preparation of the compositions provided herein include any such carriers known to those skilled in the art to be suitable for the particular mode of administration.

The resulting composition may be a lotion, a solution, a suspension, an emulsion or the like and is formulated as creams, gels, ointments, emulsions, solutions, elixirs, lotions, suspensions, tinctures, pastes, foams, aerosols, sprays, patches, foams or any other formulation suitable for topical administration. The preferred compositions are the cream, the gel and the lotion.

Pharmaceutical carriers or vehicles suitable for administration of the compounds provided herein include any such carriers known to those skilled in the art to be suitable for the particular mode of administration. In addition, the compounds may be formulated as the sole pharmaceutically active ingredient in the composition or may be combined with other active ingredients. The active agents are included in the carrier in an amount sufficient to exert a therapeutically useful effect i.e., amelioration of the symptoms of the skin disorder, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include lotions, creams, solutions, foam, gels, tapes and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

In some embodiment, at least one of the active agents in the combination composition of this invention is encapsulated. In one embodiment, all active agents (roflumilast, roflumilast N-oxide, or pharmaceutically acceptable salt, retinoid and BPO) are encapsulated. In one embodiment, the roflumilast, roflumilast N-oxide, or pharmaceutically acceptable salt is encapsulated. In one embodiment, the retinoid is encapsulated. In one embodiment, the BPO is encapsulated. In one embodiment, the retinoid and BPO are encapsulated (each and together).

According to some embodiments of the present invention, the coated/encapsulated form of the active agent(s) (microcapsule) may be in form of a polymeric microsponge/silica microsphere where the active agent is adsorbed, embedded, impregnated or entrapped in the microsponge/silica microsphere as described for example in U.S. Pat. Nos. 4,690,825; 5,145,675, 5,879,716, 5,955,109, and 9,452,137 incorporated herein by reference in their entirety.

In other embodiments, microcapsules are formed by the encapsulation process disclosed in the following publications (herein incorporated by reference): U.S. Pat. Nos. 7,629,394, 9,205,395, US 2015/0328615, US 2014/0186630. Controlled release microcapsules: IN01958CH2007, IN02080CH2007, U.S. Pat. Nos. 4,235,872, 4,670,250, EP 0248531, U.S. Pat. Nos. 4,970,031, 5,238,714, WO9321764, U.S. Pat. No. 5,575,987, WO9420075, US 2004/137031, US 2006/003014, US 2010/180464; which are incorporated herein by reference in their entirety.

### Methods of Treatment

According to an aspect of the invention, there is provided a method of treatment of a skin disorder selected from acne and rosacea, by treatment of a subject in need thereof with a combination composition of roflumilast, roflumilast N-oxide or pharmaceutically acceptable salt thereof and at least one additional active agent.

In some embodiments, the effective amount is a therapeutically effective amount of roflumilast, roflumilast N-oxide or pharmaceutically acceptable salt thereof and at least one additional active agent, namely an amount which will cure, treat, alleviate or prevent a skin disorder selected from acne and rosacea.

According to an aspect of the invention, there is provided a method of treating hidradenitis suppurativa in a subject, the method comprises administering a topical composition comprising roflumilast, roflumilast N-oxide or pharmaceutically acceptable salt thereof. In another embodiment, the concentration of the roflumilast, roflumilast N-oxide or pharmaceutically acceptable salt thereof is between about 0.01% to 1% w/w. In another embodiment, the roflumilast, roflumilast N-oxide or pharmaceutically acceptable salt thereof is the sole active agent.

According to an aspect of the invention, there is provided a method of treating prurigo nodularis in a subject, the method comprises administering a topical composition comprising roflumilast, roflumilast N-oxide or pharmaceutically acceptable salt thereof. In another embodiment, the concentration of the roflumilast, roflumilast N-oxide or pharmaceutically acceptable salt thereof is between about 0.01% to 1% w/w. In another embodiment, the roflumilast, roflumilast N-oxide or pharmaceutically acceptable salt thereof is the sole active agent.

In some embodiments, co-administration of roflumilast, roflumilast N-oxide or pharmaceutically acceptable salt thereof and at least one additional active agent selected from a retinoid and benzoyl peroxide (BPO) and combinations thereof, provides an additive and/or synergistic effect while treating, preventing or alleviating a skin disorder.

In some other embodiments, the co-administration may be made either by administration of a single combination composition, or alternatively by separate administration of a first composition comprising roflumilast, roflumilast N-oxide or pharmaceutically acceptable salt thereof and optionally a retinoid and a second composition comprising BPO, or alternatively by separate administration of a first composition comprising roflumilast, roflumilast N-oxide or pharmaceutically acceptable salt thereof and a second composition comprising BPO and optionally a retinoid, or alternatively by separate administration of a first composition comprising roflumilast, roflumilast N-oxide or pharmaceutically acceptable salt thereof and a second composition comprising retinoid and optionally a BPO or alternatively by separate administration of a first composition comprising roflumilast, roflumilast N-oxide or pharmaceutically acceptable salt thereof; a second composition comprising BPO and a third composition comprising retinoid; wherein the compositions are administered concurrently or sequentially.

### Regimen of Administration of the Topical Combination Compositions Comprising Roflumilast and at Least One Additional Active Agent

Therapeutically effective concentrations of the active agents in the compositions of this invention for treatment, prevention or alleviation of the symptoms manifested by a skin disorder are determined by empirical methods known in the art.

The concentration of the active agents in the composition will depend on absorption, inactivation, excretion rates of the active compound, synergistic and/or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of roflumilast and the additional active agent in the developed or marketed single drug currently being developed or used for the treatment of a skin disorder. The dosage and regimen of administration may be determined by dose finding studies, as known in the art.

Exemplary dosages, strengths and concentrations of roflumilast, roflumilast N-oxide or pharmaceutically acceptable salt thereof in the topical combination compositions of this invention, are in the range of from about or at 0.1%, 0.25%, 0.5%, 1%, 2% or 3% w/w. Typical strengths in the topical combination compositions of this invention are 0.25%, 0.5%, 1% or 3% w/w roflumilast.

Exemplary strengths and concentrations of BPO in the topical combination compositions comprising BPO are 2%, 3%, 4%, 5%, 6%, 7%, 8%, 9% or 10% w/w. Typical strengths in the topical combination compositions of this invention are 2%, 5%, or 10% w/w.

Exemplary strengths and concentrations of the least one retinoid in the topical combination compositions are 0.01%, 0.25%, 0.05%, 0.1%, 0.2%, 0.3%, 0.4% or 0.5% w/w. Typical strengths in the topical combination compositions of this invention are 0.05%, or 0.1% w/w.

The frequency of administration can be determined empirically. Exemplary frequencies are once daily, twice daily, weekly, bi-weekly or monthly.

Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

### Kits

Kits containing the combination compositions optionally including instructions for administration are provided. The combinations include, for example, the compositions as provided herein. Additionally, provided herein are kits containing the above-described combinations and optionally instructions for administration by topical, transdermal, or other routes, depending on the composition to be delivered.

The compositions provided herein can be packaged as articles of manufacture containing packaging material, a composition provided herein, and a label that indicates that the composition is for treating a skin disorder, and is formulated for topical delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

### EMBODIMENTS

In some embodiments, there is provided a topical composition comprising from about 0.25% w/w to about 3.0% w/w roflumilast, roflumilast N-oxide or pharmaceutically acceptable salt thereof, and at least one additional active agent selected from about 0.001% w/w to about 0.5% w/w of at least one retinoid selected from tretinoin, trifarotene and adapalene, from about 2% w/w to about 10% w/w benzoyl peroxide (BPO) and combinations thereof, and a carrier suitable for topical administration.

In some embodiments, there is provided a topical composition comprising from about 0.25% w/w to about 3.0% w/w roflumilast, roflumilast N-oxide or pharmaceutically acceptable salt thereof, and at least one additional active agent selected from about 0.001% w/w to about 0.5% w/w of tazarotene, from about 2% w/w to about 10% w/w benzoyl peroxide (BPO) and combinations thereof, and a carrier suitable for topical administration, wherein the carrier does not include hexylene glycol or diethylene glycol monoethyl ether.

In some embodiments, there is provided a topical composition comprising from about 0.25% w/w to about 3.0% w/w roflumilast, roflumilast N-oxide or pharmaceutically acceptable salt thereof, and at least one additional active agent selected from about 0.001% w/w to about 0.5% w/w of at least one retinoid selected from tretinoin, trifarotene, adapalene and tazarotene, from about 2% w/w to about 10% w/w benzoyl peroxide (BPO) and combinations thereof, and a carrier suitable for topical administration, wherein all the active agents, comprising roflumilast, roflumilast N-oxide or pharmaceutically acceptable salt thereof, and at least one additional active agent selected from at least one retinoid and benzoyl peroxide, are encapsulated.

In some embodiments, there is provided a topical composition comprising from about 0.25% w/w to about 3.0% w/w roflumilast, roflumilast N-oxide or pharmaceutically acceptable salt thereof, and at least one additional active agent selected from about 0.001% w/w to about 0.5% w/w of at least one retinoid selected from tretinoin, trifarotene, adapalene and tazarotene, from about 2% w/w to about 10% w/w benzoyl peroxide (BPO) and combinations thereof, and a carrier suitable for topical administration, wherein at least one of the active agents is encapsulated.

In some embodiments, there is provided a topical composition comprising from about 0.25% w/w to about 3.0% w/w roflumilast, roflumilast N-oxide or pharmaceutically acceptable salt thereof, and at least one additional active agent selected from about 0.001% w/w to about 0.5% w/w of at least one retinoid selected from tretinoin, trifarotene, adapalene and tazarotene, from about 2% w/w to about 10% w/w benzoyl peroxide (BPO) and combinations thereof and a carrier suitable for topical administration, wherein the active agents are fully dissolved or partly dissolved and partly suspended in the carrier suitable for topical administration comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof.

In some embodiments, there is provided a dosage form comprising a composition of this disclosure, wherein said composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe.

In some embodiments, there is provided a method of treatment, prevention or alleviation of a skin disorder selected from acne and rosacea, the method comprises topical administration to the affected area of a subject with the skin disorder a therapeutically effective amount of a composition comprising from about 0.25% w/w to about 3.0% w/w roflumilast, N-oxide or pharmaceutically acceptable salt thereof, and at least one additional active agent selected from about 0.001% w/w to about 0.5% w/w of at least one retinoid, from about 2% w/w to about 10% w/w benzoyl peroxide (BPO) and combinations thereof, and a carrier suitable for topical administration.

In some embodiments, there is provided a method of treatment, prevention or alleviation of a skin disorder selected from acne and rosacea, the method comprises topical administration to the affected area of a subject with the skin disorder a therapeutically effective amount of a composition comprising from about 0.25% w/w to about 3.0% w/w roflumilast, roflumilast N-oxide or pharmaceutically acceptable salt thereof, and at least one additional active agent selected from about 0.001% w/w to about 0.5% w/w of at least one retinoid, from about 2% w/w to about 10% w/w benzoyl peroxide (BPO) and combinations thereof, and a carrier suitable for topical administration, wherein the skin disorder is acne, selected from acne vulgaris, papulopustular acne and nodular acne.

In some embodiments, there is provided a method of treatment, prevention or alleviation of a skin disorder selected from acne and rosacea, the method comprises topical administration to the affected area of a subject with the skin disorder a therapeutically effective amount of a composition comprising from about 0.25% w/w to about 3.0% w/w roflumilast, roflumilast N-oxide or pharmaceutically acceptable salt thereof and at least one additional active agent selected from about 0.001% w/w to about 0.5% w/w of at least one retinoid, from about 2% w/w to about 10% w/w benzoyl peroxide (BPO) and combinations thereof and a carrier suitable for topical administration, wherein the skin disorder is rosacea, selected from erythematotelangiectatic rosacea, papulopustular rosacea, rhinophyma rosacea and ocular rosacea.

In some embodiments, there is provided a method of treatment, prevention or alleviation of a skin disorder selected from acne and rosacea, wherein the treatment comprises once daily or twice daily topical administration to a subject in need thereof of a therapeutically effective amount of a composition comprising from about 0.25% w/w to about 3.0% w/w roflumilast, roflumilast N-oxide or pharmaceutically acceptable salt thereof, and at least one additional active agent selected from about 0.001% w/w to about 0.5% w/w of at least one retinoid, from about 2% w/w to about 10% w/w benzoyl peroxide (BPO) and combinations thereof, and a carrier suitable for topical administration.

In some embodiments, there is provided a method of treatment, prevention or alleviation of a skin disorder selected from acne and rosacea, the method comprises topical administration to the affected area of a subject with the skin disorder a therapeutically effective amount of a composition comprising from about 0.25% w/w to about 3.0% w/w roflumilast, roflumilast N-oxide or pharmaceutically acceptable salt thereof, and at least one additional active agent selected from about 0.001% w/w to about 0.5% w/w of at least one retinoid, from about 2% w/w to about 10% w/w benzoyl peroxide (BPO) and combinations thereof, and a carrier suitable for topical administration, wherein roflumilast and the at least one additional active agent exhibit an additive or synergistic effect, thereby allowing to reduce the amounts of the active agents in the composition.

In some embodiments, there is provided a regimen of administration comprising the once daily or twice daily administration to an affected area of a subject with a skin disorder a therapeutically effective dose of the composition of a composition comprising from about 0.25% w/w to about 3.0% w/w roflumilast, roflumilast N-oxide or pharmaceutically acceptable salt thereof, and at least one additional active agent selected from about 0.001% w/w to about 0.5% w/w of at least one retinoid, from about 2% w/w to about 10% w/w benzoyl peroxide (BPO) and combinations thereof, and a carrier suitable for topical administration, until the skin disorder is cured, prevented or alleviated.

In some embodiments, there is provided a regimen of administration comprising the once daily or twice daily administration to a subject with a skin disorder a therapeutically effective amount of a dosage form comprising a composition of this disclosure, wherein said composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe.

In some embodiments, there is provided a kit comprising one or more dosage forms of this disclosure and instructions for use.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or“treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10/o.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

In the examples below, all % values referring to a solution are in (w/w).

All % values, referring to dispersions (suspensions) are in (w/w).

Unless otherwise indicated, all solutions used in the example below refer to an aqueous solution of the indicated ingredient.

Table 1 provides exemplary active agents' combinations of active agent classes and specific active agents of this invention.

All the exemplary combinations of specific active agents comprise finely milled or micronized roflumilast, at least one additional active agent selected from a retinoid (selected from tretinoin, trifarotene, adapalene and tazarotene), benzoyl peroxide (BPO) and combinations thereof.

Roflumilast and the at least one retinoid in the exemplary compositions may be used as such, as detailed in Examples 1-5 or encapsulated, as detailed in Examples 6-11.

Benzoyl peroxide, whenever present in the compositions, is encapsulated.

All active agents in the combination compositions of Examples 6-11 are SiO₂-encapsulated.

### Examples

### Example 1

General Procedure for the Preparation of a Combination Composition comprising 0.25-3% w/w non-encapsulated Roflumilast and 0.001-0.5% w/w at least one non-encapsulated Retinoid selected from Tretinoin, Trifarotene, Adapalene and Tazarotene

TABLE 2
Combination Composition comprising 0.25-3% w/w
non-encapsulated Roflumilast and 0.001-0.5% w/w
at least one non-encapsulated Retinoid selected from
Tretinoin, Trifarotene, Adapalene and Tazarotene

| Ingredient | % in formulation |
|---|---|
| Roflumilast | 0.25-3.0 |
| Retinoid(s) | 0.001-0.5 |
| DMSO | 70 |
| Propylene glycol | 22.99-25.975 |
| 2-phenoxyethanol | 0.5 |
| Methylparaben | 0.25 |
| Carbopol 980 | 3.0 |

Procedure:
Roflumilast and Retinoid(s) are dissolved in DMSO at 40° C.
Methylparaben is added under stirring
Carbopol is added under stirring
2-phenoxyethanol is dissolved in propylene glycol and added
The formulation is stirred and homogenized to obtain a homogeneous gel

The active agents in the compositions are fully dissolved or partly dissolved and partly suspended.

### Example 2

### Preparation of a Combination Composition Comprising 0.25% Roflumilast and 0.1% w/w Tretinoin

TABLE 3
Combination Composition comprising 0.25%
Roflumilast and 0.1% w/w Tretinoin

| Ingredient | % in formulation |
|---|---|
| Roflumilast | 2.0 |
| Tretinoin | 0.1 |
| DMSO | 70 |
| Propylene glycol | 24.15 |
| 2-phenoxyethanol | 0.5 |
| Methylparaben | 0.25 |
| Carbopol 980 | 3.0 |

Procedure:
Roflumilast and Tretinoin was dissolved in DMSO at 40° C.
Methylparaben is added under stirring
Carbopol is added under stirring
2-phenoxyethanol is dissolved in propylene glycol and added
The formulation is stirred and homogenized to obtain a homogeneous gel

### Example 3

### Preparation of a Combination Composition Comprising 0.25% Roflumilast and 0.0025% w/w Trifarotene

TABLE 4
Combination Composition comprising 0.25%
Roflumilas tand 0.0025% w/w Trifarotene

| Ingredient | % in formulation |
|---|---|
| Roflumilast | 0.25 |
| Trifarotene | 0.0025 |
| DMSO | 70 |
| Propylene glycol | 25.4975 |
| 2-phenoxyethanol | 0.5 |
| Methylparaben | 0.25 |
| Carbopol 980 | 3.0 |

### Procedure:

Roflumilast and Trifarotene are dissolved in DMSO at 40° C.
Methylparaben is added under stirring
Carbopol is added under stirring
2-phenoxyethanol is dissolved in propylene glycol and added
The formulation is stirred and homogenized to obtain a homogeneous gel

### Example 4

### Preparation of a Combination Composition Comprising 0.25% Roflumilast and 0.1% w/w Adapalene

TABLE 5
Combination Composition comprising 0.25%
Roflumilast and 0.1% w/w Adapalene

| Ingredient | % in formulation |
|---|---|
| Roflumilast | 0.25 |
| Adapalene | 0.1 |
| DMSO | 70 |
| Propylene glycol | 25.9 |
| 2-phenoxyethanol | 0.5 |
| Methylparaben | 0.25 |
| Carbopol 980 | 3.0 |

### Procedure:

Roflumilast and Adapalene are dissolved in DMSO at 40° C.
Methylparaben is added under stirring
Carbopol is added under stirring
2-phenoxyethanol is dissolved in propylene glycol and added
The formulation is stirred and homogenized to obtain a homogeneous gel

### Example 5

### Preparation of a Combination Composition Comprising 0.25% Roflumilast and 0.1% w/w Tazarotene

TABLE 6
Combination Composition comprising 0.25%
Roflumilast and 0.1% w/w Tazarotene

| Ingredient | % in formulation |
|---|---|
| Roflumilast | 0.25 |
| Tazarotene | 0.1 |
| DMSO | 70 |
| Propylene glycol | 25.9 |
| 2-phenoxyethanol | 0.5 |
| Methylparaben | 0.25 |
| Carbopol 980 | 3.0 |

Procedure:
Roflumilast and Tazarotene are dissolved in DMSO at 40° C.
Methylparaben is added under stirring
Carbopol is added under stirring
2-phenoxyethanol is dissolved in propylene glycol and added
The formulation is stirred and homogenized to obtain a homogeneous gel

### Example 6

### General Procedure for the Preparation of a Cream Combination Composition Comprising 0.25-3% w/w SiO₂-Encapsulated Roflumilast (E-Roflumilast) and 0.001-0.5% w/w at Least One SiO₂-Encapsulated Retinoid (E-Retinoid) Selected from Tretinoin, Trifarotene, Adapalene and Tazarotene

General Procedure:

The general procedure of Examples 6-11 comprises three stages:
1. Preparation of 3.06% w/w encapsulated retinoid (E-Retinoid)
2. Preparation of 15% w/w encapsulated roflumilast (E-Roflumilast)
3. Preparation of Cream Formulation of 0.001-0.5% w/w Encapsulated Retinoid and 0.25-3% w/w Encapsulated Roflumilast from #1 and #2 in the desired proportions, diluted as detailed in Stage 3 below.
   Stage-1 Preparation of Encapsulated Retinoid (Selected from Tretinoin, Trifarotene, Adapalene and Tazarotene), 3.06% (E-Retinoid)

### a) Oil Phase

8.62 grams of Butylated hydroxyl toluene (BHT) and 45.9 grams of retinoid are mixed in 129.3 grams of Squalane. 86.16 grams of Tetraethoxysilane (TEOS) are added, and the resulted mixture is milled at 5000 rpm in a ball mill for 10 minutes with an upper propeller mixer at a speed of 250 rpm for 7 minutes, followed by 400 rpm for 3 minutes. 140.4 grams of milled retinoid in oil is aliquoted out and then heated to 60° C. 9.0 grams of Beeswax are added and melted in the oil phase.

### b) Water Phase

3.3 grams CTAC (Cetrimonium Chloride) are dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) is added to the water phase and homogenized at 4000 rpm for 1 minute. 17.9 grams of sodium silicate extra pure solution (28%) are added to the emulsion. The pH of the emulsion is adjusted to 3.0-5.0 using HCl 5N solution. Water is added to complete the total weight of the mixture to 650 grams. The suspension is then stirred for 17 hours at 25° C. for the TEOS hydrolysis to be completed. The composition of the final encapsulated retinoid water suspension product is shown in Table 7.

TABLE 7
Composition of an encapsulated retinoid
3.06% water suspension

| | % of pure ingredient |
|---|---|
| Ingredient | in the suspension |
| Beeswax | 1.15 |
| Squalane | 8.62 |
| TEOS | 5.74 |
| Retinoid | 3.06 |
| Cetrimonium Chloride | 0.15 |
| Sodium hydroxide | 0.74 |
| Hydrochloric acid | 0.40 |
| Butylated hydroxytoluene | 0.57 |
| Sterile Water for Irrigation | 79.56 |

### Stage-2 Preparation of Encapsulated Roflumilast, 15% (E-Roflumilast Water Suspension)

### a) Preparation of Roflumilast Suspension and Acid Cocktail

Roflumilast suspension is prepared by mixing 125.67 grams of CTAC CT-429 (Cetrimonium Chloride 30%), 2250 grams of Roflumilast, and 5200 grams water under high shear. The solution is homogenized for 60 minutes at 33° C. (no more than 45° C.), and then the pH of the solution is adjusted to 7.0 using sodium hydroxide solution (20).

An acid cocktail is prepared using 493 grams Hydrochloric acid (37%), 98 grams anhydrous Citric Acid, 147 grams Lactic Acid (90%), and 794 grams water.

### b) Coating Cycle

The coating cycle is started by adding 38 grams sodium silicate solution extra pure (28%) to the Roflumilast suspension prepared in step a) under high shear, followed by adding the acid cocktail prepared in step (a) to adjust the pH to be lower than 6.8, and followed by adding 57 grams PDAC (3%) solution to the mixture. The cycle is repeated 50 times while the mixture is stirred under high shear for 17 hours. After the 50 cycles, the pH of the mixture is adjusted to 5.0 using the acid cocktail, and water is added to complete the total weight of the mixture to 15 kilograms. The composition of the final Roflumilast water suspension product is shown in Table 8.

TABLE 8
Composition of the encapsulated Roflumilast
15% water suspension

| | % of ingredient |
|---|---|
| Ingredient | in the suspension |
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Roflumilast | 15.00 |
| Sterile Water for Irrigation | 78.83 |

### Stage-3 Preparation of Cream Formulation of Encapsulated Retinoid and Encapsulated Roflumilast

Oil Phase: 720.0 grams of Cyclomethicone 5-N, 540.0 of grams Cetyl Alcohol, 360.0 grams Polyoxyl 100 Stearate, and 540.0 grams of Glyceryl Monostearate are mixed at 70° C.

Water phase: 18.0 grams of Ethylenediaminetetraacetate Disodium salt are dissolved in 6500 grams of water. 720.0 grams of glycerin (99.5%) are added to the solution. After the solution is heated to 70° C., 72.0 grams of Carbopol 980 NF are added, and the resulting mixture is homogenized at 3300 rpm for 10 minutes to ensure that all materials completely melted and dissolved. 76.5 grams of sodium hydroxide (20%) are then added and the mixture is stirred under high shear for 10 minutes at no less than 70° C.

The oil phase is added to the water phase under high shear at 78° C., and the resulting emulsion is homogenized at 3300 rpm for 10 minutes.

300-3580 grams of encapsulated Roflumilast 15% water suspension made as described above is added to the emulsion at 65° C. and mixed at 1400 rpm for 10 minutes. The emulsion is cooled to 35° C. and 5.9-2940 grams of encapsulated retinoid 3.06% water suspension made as described above are added, and the emulsion is stirred at 1400 rpm for 10 minutes. Water is added until the total weight of the cream reached 18 kilograms. The composition of the formulation prepared in this example is shown in Table 9.

TABLE 9
Composition of Cream Formulation of Encapsulated
Retinoid and Encapsulated Roflumilast

| | % of pure ingredient |
|---|---|
| Ingredient | in the composition |
| Polyquarternium-7 | 0.21 |
| Hydrochloric Acid | 0.51 |
| Citric Acid, Anhydrous | 0.18 |
| Lactic Acid | 0.25 |
| Silicon Dioxide | 1.44 |
| Sodium hydroxide | 0.09 |
| Cetrimonium Chloride | 0.11 |
| Roflumilast | 0.25-3.0 |
| Beeswax | 0.04 |
| Squalane | 0.28 |
| Ethanol (Alcohol) | 0.14 |
| Retinoid | 0.001-0.5 |
| Butylated hydroxytoluene | 0.02 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Di sodium | 0.10 |
| Carbopol 980 | 0.40 |
| Sterile Water for Irrigation | Up to 100% |

### Example 7

### Preparation of a Combination Composition Comprising 0.25% w/w SiO₂-Encapsulated Roflumilast and 0.1% w/w SiO₂-Encapsulated Tretinoin

### Preparation of Encapsulated Tretinoin, 3.06% (E-ATRA)

### a) Oil Phase

8.62 grams of Butylated hydroxyl toluene (BHT) and 45.9 grams of ATRA are mixed in 129.3 grams of Squalane. 86.16 grams of Tetraethoxysilane (TEOS) are added, and the resulted mixture is milled at 5000 rpm in a ball mill for 10 minutes with an upper propeller mixer at a speed of 250 rpm for 7 minutes, followed by 400 rpm for 3 minutes. 140.4 grams of milled tretinoin in oil is aliquoted out and then heated to 60° C. 9.0 grams of Beeswax are added and melted in the oil phase.

### b) Water Phase

3.3 grams CTAC (Cetrimonium Chloride) are dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) is added to the water phase and homogenized at 4000 rpm for 1 minute. 17.9 grams of sodium silicate extra pure solution (28%) are added to the emulsion. The pH of the emulsion is adjusted to 3.0-5.0 using HCl 5N solution. Water is added to complete the total weight of the mixture to 650 grams. The suspension is then stirred for 17 hours at 25° C. for the TEOS hydrolysis to be completed. The composition of the final encapsulated ATRA water suspension product is shown in Table 10.

TABLE 10
Composition of the encapsulated ATRA
3.06% water suspension

| | % of pure ingredient |
|---|---|
| Ingredient | in the suspension |
| Beeswax | 1.15 |
| Squalane | 8.62 |
| TEOS | 5.74 |
| tretinoin | 3.06 |
| Cetrimonium Chloride | 0.15 |
| Sodium hydroxide | 0.74 |
| Hydrochloric acid | 0.40 |
| Butylated hydroxytoluene | 0.57 |
| Sterile Water for Irrigation | 79.56 |

### Preparation of Encapsulated Roflumilast, 15% (E-Roflumilast Water Suspension)

### a) Preparation of Roflumilast Suspension and Acid Cocktail

Roflumilast suspension is prepared by mixing 125.67 grams of CTAC CT-429 (Cetrimonium Chloride 30%), 2250 grams of Roflumilast, and 5200 grams water under high shear. The solution is homogenized for 60 minutes at 33° C. (no more than 45° C.), and then the pH of the solution is adjusted to 7.0 using sodium hydroxide solution (20).

An acid cocktail is prepared using 493 grams Hydrochloric acid (37%), 98 grams anhydrous Citric Acid, 147 grams Lactic Acid (90%), and 794 grams water.

### b) Coating Cycle

The coating cycle is started by adding 38 grams sodium silicate solution extra pure (28%) to the Roflumilast suspension prepared in step a) under high shear, followed by adding the acid cocktail prepared in step (a) to adjust the pH to be lower than 6.8, and followed by adding 57 grams PDAC (3%) solution to the mixture. The cycle is repeated 50 times while the mixture is stirred under high shear for 17 hours. After the 50 cycles, the pH of the mixture is adjusted to 5.0 using the acid cocktail, and water is added to complete the total weight of the mixture to 15 kilograms. The composition of the final Roflumilast water suspension product is shown in Table 11.

TABLE 11
Composition of encapsulated Roflumilast
15% water suspension

| | % of ingredient |
|---|---|
| Ingredient | in the suspension |
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Roflumilast | 15.00 |
| Sterile Water for Irrigation | 78.83 |

### Preparation of Cream Formulation of 0.1% Encapsulated Tretinoin and 0.25% Encapsulated Roflumilast

Oil Phase: 720.0 of grams Cyclomethicone 5-N, 540.0 of grams Cetyl Alcohol, 360.0 grams Polyoxyl 100 Stearate, and 540.0 grams of Glyceryl Monostearate are mixed at 70° C.

Water phase: 18.0 grams of Ethylenediaminetetraacetate Disodium salt are dissolved in 6500 grams of water. 720.0 grams of glycerin (99.5%) are added to the solution. After the solution is heated to 70° C., 72.0 grams of Carbopol 980 NF are added, and the resulting mixture is homogenized at 3300 rpm for 10 minutes to ensure that all materials completely melted and dissolved. 76.5 grams of sodium hydroxide (20%) are then added and the mixture is stirred under high shear for 10 minutes at no less than 70° C.

The oil phase is added to the water phase under high shear at 78° C., and the resulting emulsion is homogenized at 3300 rpm for 10 minutes. 300 grams of encapsulated Roflumilast 15% water suspension made as described above is added to the emulsion at 65° C. and mixed at 1400 rpm for 10 minutes. The emulsion is cooled to 35° C. and 589 grams of encapsulated tretinoin 3.06% water suspension made as described above are added, and the emulsion is stirred at 1400 rpm for 10 minutes. Water is added until the total weight of the cream reached 18 kilograms. The composition of the formulation prepared in this example is shown in Table 12.

TABLE 12
Composition of Cream Formulation comprising
0.1% Encapsulated Tretinoin and 0.25%
Encapsulated Roflumilast

| | % of pure ingredient |
|---|---|
| Ingredient | in the composition |
| Polyquarternium-7 | 0.21 |
| Hydrochloric Acid | 0.51 |
| Citric Acid, Anhydrous | 0.18 |
| Lactic Acid | 0.25 |
| Silicon Dioxide | 1.44 |
| Sodium hydroxide | 0.09 |
| Cetrimonium Chloride | 0.11 |
| Roflumilast | 0.25 |
| Beeswax | 0.04 |
| Squalane | 0.28 |
| Ethanol (Alcohol) | 0.14 |
| Tretinoin | 0.1 |
| Butylated hydroxytoluene | 0.02 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Di sodium | 0.10 |
| Carbopol 980 | 0.40 |
| Sterile Water for Irrigation | Up to 100% |

### Example 8

### Preparation of a Combination Composition Comprising 0.25% w/w SiO₂-Encapsulated Roflumilast and 0.0025% w/w SiO₂-Encapsulated Trifarotene

### Preparation of Encapsulated Trifarotene, 3.06% (E-Trifarotene)

### a) Oil Phase

8.62 grams of Butylated hydroxy toluene (BHT) and 45.9 grams of Trifarotene are mixed in 129.3 grams of Squalane. 86.16 grams of Tetraethoxysilane (TEOS) are added, and the resulted mixture is milled at 5000 rpm in a ball mill for 10 minutes with an upper propeller mixer at a speed of 250 rpm for 7 minutes, followed by 400 rpm for 3 minutes. 140.4 grams of milled Trifarotene in oil is aliquoted out and then heated to 60° C. 9.0 grams of Beeswax are added and melted in the oil phase.

### b) Water Phase

3.3 grams CTAC (Cetrimonium Chloride) are dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) is added to the water phase and homogenized at 4000 rpm for 1 minute. 17.9 grams of sodium silicate extra pure solution (28%) are added to the emulsion. The pH of the emulsion is adjusted to 3.0-5.0 using HCl 5N solution. Water is added to complete the total weight of the mixture to 650 grams. The suspension is then stirred for 17 hours at 25° C. for the TEOS hydrolysis to be completed. The composition of the final encapsulated Trifarotene water suspension product is shown in Table 13.

TABLE 13
Composition of encapsulated Trifarotene
3.06% water suspension

| | % of pure ingredient |
|---|---|
| Ingredient | in the suspension |
| Beeswax | 1.15 |
| Squalane | 8.62 |
| TEOS | 5.74 |
| Trifarotene | 3.06 |
| Cetrimonium Chloride | 0.15 |
| Sodium hydroxide | 0.74 |
| Hydrochloric acid | 0.40 |
| Butylated hydroxytoluene | 0.57 |
| Sterile Water for Irrigation | 79.56 |

### Preparation of Encapsulated Roflumilast, 15% (E-Roflumilast Water Suspension)

### a) Preparation of Roflumilast Suspension and Acid Cocktail

Roflumilast suspension is prepared by mixing 125.67 grams of CTAC CT-429 (Cetrimonium Chloride 30%), 2250 grams of Roflumilast, and 5200 grams water under high shear. The solution is homogenized for 60 minutes at 33° C. (no more than 45° C.), and then the pH of the solution is adjusted to 7.0 using sodium hydroxide solution (20).

An acid cocktail is prepared using 493 grams Hydrochloric acid (37%), 98 grams anhydrous Citric Acid, 147 grams Lactic Acid (90%), and 794 grams water.

### b) Coating Cycle

The coating cycle is started by adding 38 grams sodium silicate solution extra pure (28%) to the Roflumilast suspension prepared in step a) under high shear, followed by adding the acid cocktail prepared in step (a) to adjust the pH to be lower than 6.8, and followed by adding 57 grans PDAC (3%) solution to the mixture. The cycle is repeated 50 times while the mixture is stirred under high shear for 17 hours. After the 50 cycles, the pH of the mixture is adjusted to 5.0 using the acid cocktail, and water is added to complete the total weight of the mixture to 15 kilograms. The composition of the final Roflumilast water suspension product is shown in Table 14.

TABLE 14
Composition of encapsulated Roflumilast
15% water suspension

| | % of ingredient |
|---|---|
| Ingredient | in the suspension |
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Roflumilast | 15.00 |
| Sterile Water for Irrigation | 78.83 |

### Preparation of Cream Formulation of 0.0025% Encapsulated Trifarotene and 0.25% Encapsulated Roflumilast

Oil Phase: 720.0 of grams Cyclomethicone 5-N, 540.0 of grams Cetyl Alcohol, 360.0 grams Polyoxyl 100 Stearate, and 540.0 grams of Glyceryl Monostearate are mixed at 70° C.

Water phase: 18.0 grams of Ethylenediaminetetraacetate Disodium salt are dissolved in 6500 grams of water. 720.0 grams of glycerin (99.5%) are added to the solution. After the solution is heated to 70° C., 72.0 grams of Carbopol 980 NF are added, and the resulting mixture is homogenized at 3300 rpm for 10 minutes to ensure that all materials completely melted and dissolved. 76.5 grams of sodium hydroxide (20%) are then added and the mixture is stirred under high shear for 10 minutes at no less than 70° C.

The oil phase is added to the water phase under high shear at 78° C., and the resulting emulsion is homogenized at 3300 rpm for 10 minutes. 300 grams of encapsulated Roflumilast 15% water suspension made as described above is added to the emulsion at 65° C. and mixed at 1400 rpm for 10 minutes. The emulsion is cooled to 35° C. and 14.7 grams of encapsulated Trifarotene 3.06% water suspension made as described above are added, and the emulsion is stirred at 1400 rpm for 10 minutes. Water is added until the total weight of the cream reached 18 kilograms. The composition of the formulation prepared in this example is shown in Table 15.

TABLE 15
Composition of Cream Formulation comprising
0.0025% Encapsulated Trifarotene and
0.25% Encapsulated Roflumilast

| | % of pure ingredient |
|---|---|
| Ingredient | in the composition |
| Polyquarternium-7 | 0.21 |
| Hydrochloric Acid | 0.51 |
| Citric Acid, Anhydrous | 0.18 |
| Lactic Acid | 0.25 |
| Silicon Dioxide | 1.44 |
| Sodium hydroxide | 0.09 |
| Cetrimonium Chloride | 0.11 |
| Roflumilast | 0.25 |
| Beeswax | 0.04 |
| Squalane | 0.28 |
| Ethanol (Alcohol) | 0.14 |
| Trifarotene | 0.0025 |
| Butylated hydroxytoluene | 0.02 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Di sodium | 0.10 |
| Carbopol 980 | 0.40 |
| Sterile Water for Irrigation | Up to 100% |

### Example 9

### General Procedure for the Preparation of a Combination Composition Comprising 0.25-3% w/w SiO₂-Encapsulated Roflumilast, 0.001-0.5% w/w at Least One SiO₂-Encapsulated Retinoid Selected from Tretinoin, Trifarotene, Adapalene and Tazarotene and 2-10% w/w SiO₂-Encapsulated Benzoyl Peroxide (BPO)

Preparation of 3.06% w/w Encapsulated Retinoid (E-Retinoid, Selected from Tretinoin, Trifarotene, Adapalene and Tazarotene)

### a) Oil Phase

8.62 grams of Butylated hydroxyl toluene (BHT) and 45.9 grams of retinoid are mixed in 129.3 grams of Squalane. 86.16 grams of Tetraethoxysilane (TEOS) are added, and the resulted mixture is milled at 5000 rpm in a ball mill for 10 minutes with an upper propeller mixer at a speed of 250 rpm for 7 minutes, followed by 400 rpm for 3 minutes. 140.4 grams of milled retinoid in oil is aliquoted out and then heated to 60° C. 9.0 grams of Beeswax are added and melted in the oil phase.

### b) Water Phase

3.3 grams CTAC (Cetrimonium Chloride) are dissolved in 490.0 g water at 60° C. Unless indicated otherwise, in all examples described herein, the term “water” refers to sterile water for irrigations (USP).

### c) Core-Shell Step

124.5 grams of the oil phase prepared in step (a) is added to the water phase and homogenized at 4000 rpm for 1 minute. 17.9 grams of sodium silicate extra pure solution (28%) are added to the emulsion. The pH of the emulsion is adjusted to 3.0-5.0 using HCl 5N solution. Water is added to complete the total weight of the mixture to 650 grans. The suspension is then stirred for 17 hours at 25° C. for the TEOS hydrolysis to be completed. The composition of the final encapsulated retinoid water suspension product is shown in Table 16.

TABLE 16
Composition of encapsulated retinoid
3.06% water suspension

| | % of pure ingredient |
|---|---|
| Ingredient | in the suspension |
| Beeswax | 1.15 |
| Squalane | 8.62 |
| TEOS | 5.74 |
| retinoid | 3.06 |
| Cetrimonium Chloride | 0.15 |
| Sodium hydroxide | 0.74 |
| Hydrochloric acid | 0.40 |
| Butylated hydroxytoluene | 0.57 |
| Sterile Water for Irrigation | 79.56 |

### Preparation of Encapsulated Roflumilast, 15% (E-Roflumilast Water Suspension)

### a) Preparation of Roflumilast Suspension and Acid Cocktail

Roflumilast suspension is prepared by mixing 125.67 grams of CTAC CT-429 (Cetrimonium Chloride 30%), 2250 grams of Roflumilast, and 5200 grams water under high shear. The solution is homogenized for 60 minutes at 33° C. (no more than 45° C.), and then the pH of the solution is adjusted to 7.0 using sodium hydroxide solution (20).

An acid cocktail is prepared using 493 grams Hydrochloric acid (37%), 98 grams anhydrous Citric Acid, 147 grams Lactic Acid (90%), and 794 grams water.

### b) Coating Cycle

The coating cycle is started by adding 38 grams sodium silicate solution extra pure (28%) to the Roflumilast suspension prepared in step a) under high shear, followed by adding the acid cocktail prepared in step (a) to adjust the pH to be lower than 6.8, and followed by adding 57 grans PDAC (3%) solution to the mixture. The cycle is repeated 50 times while the mixture is stirred under high shear for 17 hours. After the 50 cycles, the pH of the mixture is adjusted to 5.0 using the acid cocktail, and water is added to complete the total weight of the mixture to 15 kilograms. The composition of the final Roflumilast water suspension product is shown in Table 17.

TABLE 17
Composition of encapsulated Roflumilast
15% water suspension

| | % of ingredient |
|---|---|
| Ingredient | in the suspension |
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Roflumilast | 15.00 |
| Sterile Water for Irrigation | 78.83 |

### Preparation of Encapsulated BPO (15% E-BPO Water Suspension)

### a) Preparation of Benzoyl Peroxide Solution and Acid Cocktail

A benzoyl peroxide (BPO) suspension is prepared by mixing 125.67 grams of CTAC CT-429 (Cetrimonium Chloride 30%), 3008 grams of hydrous benzoyl peroxide, and 5200 grams water under high shear. The solution is homogenized for 60 minutes at 33° C. (no more than 45° C.), and then the pH of the solution is adjusted to 7.0 using sodium hydroxide solution (20%).

An acid cocktail is prepared using 493 grams Hydrochloric acid (37%), 98 grams anhydrous Citric Acid, 147 grams Lactic Acid (90%), and 794 grams water.

### b) Coating Cycle

The coating cycle is started by adding 38 grams sodium silicate solution extra pure (28%) to the benzoyl peroxide solution prepared in step a) under high shear, followed by adding the acid cocktail prepared in step (a) to adjust the pH to be lower than 6.8, and followed by adding 57 grams PDAC (3%) solution to the mixture. The cycle is repeated 50 times while the mixture is stirred under high shear for 17 hours. After the 50 cycles, the pH of the mixture is adjusted to 5.0 using the acid cocktail, and water is added to complete the total weight of the mixture to 15 kilograms. The composition of the final BPO water suspension product is shown in Table 18.

TABLE 18
Composition of encapsulated BPO
15% water suspension

| | % of ingredient |
|---|---|
| Ingredient | in the suspension |
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Hydrous Benzoyl Peroxide | 15.00 |
| Sterile Water for Irrigation | 78.83 |

### Preparation of Cream Formulation Comprising Encapsulated Retinoid, Encapsulated Roflumilast and Encapsulated BPO

Oil Phase: 720.0 of grams Cyclomethicone 5-N, 540.0 of grams Cetyl Alcohol, 360.0 grams Polyoxyl 100 Stearate, and 540.0 grams of Glyceryl Monostearate are mixed at 70° C.

Water phase: 18.0 grams of Ethylenediaminetetraacetate Disodium salt are dissolved in 6500 grams of water. 720.0 grams of glycerin (99.5%) are added to the solution. After the solution is heated to 70° C., 72.0 grams of Carbopol 980 NF are added, and the resulting mixture is homogenized at 3300 rpm for 10 minutes to ensure that all materials completely melted and dissolved. 76.5 grams of sodium hydroxide (20%) are then added and the mixture is stirred under high shear for 10 minutes at no less than 70° C.

The oil phase is added to the water phase under high shear at 78° C., and the resulting emulsion is homogenized at 3300 rpm for 10 minutes. 300-3580 grams of encapsulated Roflumilast 15% water suspension made as described above is added to the emulsion at 65° C. and mixed at 1400 rpm for 10 minutes. 72.0 grams of Citric Acid and 2385-11920 grams of encapsulated BPO 15% water suspension made as described above are mixed and added to the emulsion. The emulsion is cooled to 35° C. and 5.9-2940 grams of encapsulated retinoid 3.06% water suspension made as described above are added, and the emulsion is stirred at 1400 rpm for 10 minutes. Water is added until the total weight of the cream reached 18 kilograms. The composition of the formulation prepared in this example is shown in Table 19.

TABLE 19
Composition of Cream Formulation comprising
Encapsulated Retinoid, Encapsulated Roflumilast
and Encapsulated BPO

| | % of pure ingredient |
|---|---|
| Ingredient | in the composition |
| Polyquarternium-7 | 0.42 |
| Hydrochloric Acid | 1.02 |
| Citric Acid, Anhydrous | 0.36 |
| Lactic Acid | 0.50 |
| Silicon Dioxide | 2.98 |
| Sodium hydroxide | 0.18 |
| Cetrimonium Chloride | 0.22 |
| Roflumilast | 0.25-3.0 |
| Hydrous Benzoyl Peroxide | 2.0-10.0 |
| Beeswax | 0.04 |
| Squalane | 0.28 |
| Ethanol (Alcohol) | 0.14 |
| Retinoid | 0.001-0.5 |
| Butylated hydroxytoluene | 0.02 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Di sodium | 0.10 |
| Carbopol 980 | 0.40 |
| Sterile Water for Irrigation | Up to 100% |

### Example 10

### Preparation of a Combination Composition Comprising 0.25% w/w SiO₂-Encapsulated Roflumilast, 0.1% w/w SiO₂-Encapsulated Tretinoin and 3% w/w SiO₂-Encapsulated Benzoyl Peroxide (BPO)

### Preparation of Encapsulated Tretinoin, 3.06% (E-ATRA)

### a) Oil Phase

8.62 grams of Butylated hydroxy toluene (BHT) and 45.9 grams of ATRA are mixed in 129.3 grams of Squalane. 86.16 grams of Tetraethoxysilane (TEOS) are added, and the resulted mixture is milled at 5000 rpm in a ball mill for 10 minutes with an upper propeller mixer at a speed of 250 rpm for 7 minutes, followed by 400 rpm for 3 minutes. 140.4 grams of milled tretinoin in oil is aliquoted out and then heated to 60° C. 9.0 grams of Beeswax are added and melted in the oil phase.

### b) Water Phase

3.3 grams CTAC (Cetrimonium Chloride) are dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) is added to the water phase and homogenized at 4000 rpm for 1 minute. 17.9 grams of sodium silicate extra pure solution (28%) are added to the emulsion. The pH of the emulsion is adjusted to 3.0-5.0 using HCl 5N solution. Water is added to complete the total weight of the mixture to 650 grams. The suspension is then stirred for 17 hours at 25° C. for the TEOS hydrolysis to be completed. The composition of the final encapsulated ATRA water suspension product is shown in Table 20.

TABLE 20
Composition of encapsulated
ATRA 3.06% water suspension

| | % of pure ingredient |
|---|---|
| Ingredient | in the suspension |
| Beeswax | 1.15 |
| Squalane | 8.62 |
| TEOS | 5.74 |
| Tretinoin | 3.06 |
| Cetrimonium Chloride | 0.15 |
| Sodium hydroxide | 0.74 |
| Hydrochloric acid | 0.40 |
| Butylated hydroxytoluene | 0.57 |
| Sterile Water for Irrigation | 79.56 |

### Preparation of Encapsulated Roflumilast, 15% (E-Roflumilast Water Suspension)

### a) Preparation of Roflumilast Suspension and Acid Cocktail

Roflumilast suspension is prepared by mixing 125.67 grams of CTAC CT-429 (Cetrimonium Chloride 30%), 2250 grams of Roflumilast, and 5200 grams water under high shear. The solution is homogenized for 60 minutes at 33° C. (no more than 45° C.), and then the pH of the solution is adjusted to 7.0 using sodium hydroxide solution (20%).

An acid cocktail is prepared using 493 grams Hydrochloric acid (37%), 98 grams anhydrous Citric Acid, 147 grams Lactic Acid (90%), and 794 grams water.

### b) Coating Cycle

The coating cycle is started by adding 38 grams sodium silicate solution extra pure (28%) to the Roflumilast suspension prepared in step a) under high shear, followed by adding the acid cocktail prepared in step (a) to adjust the pH to be lower than 6.8, and followed by adding 57 grams PDAC (3%) solution to the mixture. The cycle is repeated 50 times while the mixture is stirred under high shear for 17 hours. After the 50 cycles, the pH of the mixture is adjusted to 5.0 using the acid cocktail, and water is added to complete the total weight of the mixture to 15 kilograms. The composition of the final Roflumilast water suspension product is shown in Table 21.

TABLE 21
Composition of encapsulated Roflumilast
15% water suspension

| | % of ingredient |
|---|---|
| Ingredient | in the suspension |
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Roflumilast | 15.00 |
| Sterile Water for Irrigation | 78.83 |

### Preparation of Encapsulated BPO (15% E-BPO Water Suspension)

### a) Preparation of Benzoyl Peroxide Solution and Acid Cocktail

A benzoyl peroxide (BPO) suspension is prepared by mixing 125.67 grams of CTAC CT-429 (Cetrimonium Chloride 30%), 3008 grams of hydrous benzoyl peroxide, and 5200 grams water under high shear. The solution is homogenized for 60 minutes at 33° C. (no more than 45° C.), and then the pH of the solution is adjusted to 7.0 using sodium hydroxide solution (20%).

An acid cocktail is prepared using 493 grams Hydrochloric acid (37%), 98 grans anhydrous Citric Acid, 147 grams Lactic Acid (90%), and 794 grams water.

### b) Coating Cycle

The coating cycle is started by adding 38 grams sodium silicate solution extra pure (28%) to the benzoyl peroxide solution prepared in step a) under high shear, followed by adding the acid cocktail prepared in step (a) to adjust the pH to be lower than 6.8, and followed by adding 57 grans PDAC (3%) solution to the mixture. The cycle is repeated 50 times while the mixture is stirred under high shear for 17 hours. After the 50 cycles, the pH of the mixture is adjusted to 5.0 using the acid cocktail, and water is added to complete the total weight of the mixture to 15 kilograms. The composition of the final BPO water suspension product is shown in Table 22.

TABLE 22
Composition of encapsulated BPO
15% water suspension

| | % of ingredient |
|---|---|
| Ingredient | in the suspension |
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Hydrous Benzoyl Peroxide | 15.00 |
| Sterile Water for Irrigation | 78.83 |

Preparation of Cream Formulation comprising 0.1% Encapsulated Tretinoin, 0.25% Encapsulated Roflumilast and 3% encapsulated BPO

Oil Phase: 720.0 of grams Cyclomethicone 5-N, 540.0 of grams Cetyl Alcohol, 360.0 grams Polyoxyl 100 Stearate, and 540.0 grams of Glyceryl Monostearate are mixed at 70° C.

Water phase: 18.0 grams of Ethylenediaminetetraacetate Disodium salt are dissolved in 6500 grams of water. 720.0 grams of glycerin (99.5%) are added to the solution. After the solution is heated to 70° C., 72.0 grams of Carbopol 980 NF are added, and the resulting mixture is homogenized at 3300 rpm for 10 minutes to ensure that all materials completely melted and dissolved. 76.5 grams of sodium hydroxide (20%) are then added and the mixture is stirred under high shear for 10 minutes at no less than 70° C.

The oil phase is added to the water phase under high shear at 78° C., and the resulting emulsion is homogenized at 3300 rpm for 10 minutes. 300 grams of encapsulated Roflumilast 15% water suspension made as described above is added to the emulsion at 65° C. and mixed at 1400 rpm for 10 minutes. 72.0 grams of Citric Acid and 3578 grams of encapsulated BPO 15% water suspension made as described above are mixed and added to the emulsion. The emulsion is cooled to 35° C. and 589 grams of encapsulated tretinoin 3.06% water suspension made as described above are added, and the emulsion is stirred at 1400 rpm for 10 minutes. Water is added until the total weight of the cream reached 18 kilograms. The composition of the formulation prepared in this example is shown in Table 23.

TABLE 23
Composition of Cream Formulation comprising
0.1% Encapsulated Tretinoin, 0.25% Encapsulated
Roflumilast and 3% encapsulated BPO

| | % of pure ingredient |
|---|---|
| Ingredient | in the composition |
| Polyquarternium-7 | 0.42 |
| Hydrochloric Acid | 1.02 |
| Citric Acid, Anhydrous | 0.36 |
| Lactic Acid | 0.50 |
| Silicon Dioxide | 2.98 |
| Sodium hydroxide | 0.18 |
| Cetrimonium Chloride | 0.22 |
| Roflumilast | 0.25 |
| Hydrous Benzoyl Peroxide | 3.0 |
| Beeswax | 0.04 |
| Squalane | 0.28 |
| Ethanol (Alcohol) | 0.14 |
| Tretinoin | 0.1 |
| Butylated hydroxytoluene | 0.02 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Disodium | 0.10 |
| Carbopol 980 | 0.40 |
| Sterile Water for Irrigation | Up to 100% |

### Example 11

### Preparation of a Combination Composition Comprising 0.25% w/w SiO₂-Encapsulated Roflumilast, 0.0025% w/w SiO₂-Encapsulated Trifarotene and 3% w/w SiO₂-Encapsulated Benzoyl Peroxide (BPO)

### Preparation of Encapsulated Trifarotene, 3.06% (E-Trifarotene)

### a) Oil Phase

8.62 grams of Butylated hydroxy toluene (BHT) and 45.9 grams of Trifarotene are mixed in 129.3 grams of Squalane. 86.16 grams of Tetraethoxysilane (TEOS) are added, and the resulted mixture is milled at 5000 rpm in a ball mill for 10 minutes with an upper propeller mixer at a speed of 250 rpm for 7 minutes, followed by 400 rpm for 3 minutes. 140.4 grams of milled Trifarotene in oil is aliquoted out and then heated to 60° C. 9.0 grams of Beeswax are added and melted in the oil phase.

### b) Water Phase

3.3 grams CTAC (Cetrimonium Chloride) are dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) is added to the water phase and homogenized at 4000 rpm for 1 minute. 17.9 grams of sodium silicate extra pure solution (28%) are added to the emulsion. The pH of the emulsion is adjusted to 3.0-5.0 using HCl 5N solution. Water is added to complete the total weight of the mixture to 650 grams. The suspension is then stirred for 17 hours at 25° C. for the TEOS hydrolysis to be completed. The composition of the final encapsulated Trifarotene water suspension product is shown in Table 24.

TABLE 24
Composition of encapsulated Trifarotene
3.06% water suspension

| | % of pure ingredient |
|---|---|
| Ingredient | in the suspension |
| Beeswax | 1.15 |
| Squalane | 8.62 |
| TEOS | 5.74 |
| Trifarotene | 3.06 |
| Cetrimonium Chloride | 0.15 |
| Sodium hydroxide | 0.74 |
| Hydrochloric acid | 0.40 |
| Butylated hydroxytoluene | 0.57 |
| Sterile Water for Irrigation | 79.56 |

### Preparation of Encapsulated Roflumilast, 15% (E-Roflumilast Water Suspension)

### a) Preparation of Roflumilast Suspension and Acid Cocktail

Roflumilast suspension is prepared by mixing 125.67 grams of CTAC CT-429 (Cetrimonium Chloride 30%), 2250 grams of Roflumilast, and 5200 grams water under high shear. The solution is homogenized for 60 minutes at 33° C. (no more than 45° C.), and then the pH of the solution is adjusted to 7.0 using sodium hydroxide solution (20).

An acid cocktail is prepared using 493 grams Hydrochloric acid (37%), 98 grams anhydrous Citric Acid, 147 grams Lactic Acid (90%), and 794 grams water.

### b) Coating Cycle

The coating cycle is started by adding 38 grams sodium silicate solution extra pure (28%) to the Roflumilast suspension prepared in step a) under high shear, followed by adding the acid cocktail prepared in step (a) to adjust the pH to be lower than 6.8, and followed by adding 57 grams PDAC (3%) solution to the mixture. The cycle is repeated 50 times while the mixture is stirred under high shear for 17 hours. After the 50 cycles, the pH of the mixture is adjusted to 5.0 using the acid cocktail, and water is added to complete the total weight of the mixture to 15 kilograms. The composition of the final Roflumilast water suspension product is shown in Table 25.

TABLE 25
Composition of encapsulated Roflumilast
15% water suspension

| | % of ingredient |
|---|---|
| Ingredient | in the suspension |
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Roflumilast | 15.00 |
| Sterile Water for Irrigation | 78.83 |

### Preparation of Encapsulated BPO (15% E-BPO Water Suspension)

### a) Preparation of Benzoyl Peroxide Solution and Acid Cocktail

A benzoyl peroxide (BPO) suspension is prepared by mixing 125.67 grams of CTAC CT-429 (Cetrimonium Chloride 30%), 3008 grams of hydrous benzoyl peroxide, and 5200 grams water under high shear. The solution is homogenized for 60 minutes at 33° C. (no more than 45° C.), and then the pH of the solution is adjusted to 7.0 using sodium hydroxide solution (20%).

An acid cocktail is prepared using 493 grams Hydrochloric acid (37%), 98 grams anhydrous Citric Acid, 147 grams Lactic Acid (90%), and 794 grams water.

### b) Coating Cycle

The coating cycle is started by adding 38 grams sodium silicate solution extra pure (28%) to the benzoyl peroxide solution prepared in step a) under high shear, followed by adding the acid cocktail prepared in step (a) to adjust the pH to be lower than 6.8, and followed by adding 57 grams PDAC (3%) solution to the mixture. The cycle is repeated 50 times while the mixture is stirred under high shear for 17 hours. After the 50 cycles, the pH of the mixture is adjusted to 5.0 using the acid cocktail, and water is added to complete the total weight of the mixture to 15 kilograms. The composition of the final BPO water suspension product is shown in Table 26.

TABLE 26
Composition of Encapsulated BPO
15% water suspension

| | % of ingredient |
|---|---|
| Ingredient | in the suspension |
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Hydrous Benzoyl Peroxide | 15.00 |
| Sterile Water for Irrigation | 78.83 |

### Preparation of Cream Formulation Comprising 0.0025% Encapsulated Trifarotene, 0.25% Encapsulated Roflumilast and 3% Encapsulated BPO

Oil Phase: 720.0 of grams Cyclomethicone 5-N, 540.0 of grams Cetyl Alcohol, 360.0 grams Polyoxyl 100 Stearate, and 540.0 grams of Glyceryl Monostearate are mixed at 70° C.

Water phase: 18.0 grams of Ethylenediaminetetraacetate Disodium salt are dissolved in 6500 grams of water. 720.0 grams of glycerin (99.5%) are added to the solution. After the solution is heated to 70° C., 72.0 grams of Carbopol 980 NF are added, and the resulting mixture is homogenized at 3300 rpm for 10 minutes to ensure that all materials completely melted and dissolved. 76.5 grams of sodium hydroxide (20%) are then added and the mixture is stirred under high shear for 10 minutes at no less than 70° C.

The oil phase is added to the water phase under high shear at 78° C., and the resulting emulsion is homogenized at 3300 rpm for 10 minutes. 300 grams of encapsulated Roflumilast 15% water suspension made as described above is added to the emulsion at 65° C. and mixed at 1400 rpm for 10 minutes. 72.0 grams of Citric Acid and 3578 grams of encapsulated BPO 15% water suspension made as described above are mixed and added to the emulsion. The emulsion is cooled to 35° C. and 14.7 grams of encapsulated Trifarotene 3.06% water suspension made as described above are added, and the emulsion is stirred at 1400 rpm for 10 minutes. Water is added until the total weight of the cream reached 18 kilograms. The composition of the formulation prepared in this example is shown in Table 27.

TABLE 27
Composition of Cream Formulation Comprising 0.0025%
Encapsulated Trifarotene, 0.25% Encapsulated
Roflumilast and 3% encapsulated BPO

| | % of pure ingredient |
|---|---|
| Ingredient | in the composition |
| Polyquarternium-7 | 0.42 |
| Hydrochloric Acid | 1.02 |
| Citric Acid, Anhydrous | 0.36 |
| Lactic Acid | 0.50 |
| Silicon Dioxide | 2.98 |
| Sodium hydroxide | 0.18 |
| Cetrimonium Chloride | 0.22 |
| Roflumilast | 0.25 |
| Hydrous Benzoyl Peroxide | 3.0 |
| Beeswax | 0.04 |
| Squalane | 0.28 |
| Ethanol (Alcohol) | 0.14 |
| Trifarotene | 0.0025 |
| Butylated hydroxytoluene | 0.02 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Di sodium | 0.10 |
| Carbopol 980 | 0.40 |
| Sterile Water for Irrigation | Up to 100% |

What is claimed is:

1. A topical composition comprising from about 0.25% w/w to about 3.0% w/w roflumilast, roflumilast N-oxide or pharmaceutically acceptable salt thereof, at least one additional active agent selected from about 0.001% w/w to about 0.5% w/w of at least one retinoid selected from tretinoin, trifarotene and adapalene, from about 2% w/w to about 10% w/w benzoyl peroxide (BPO) and combinations thereof, and a carrier suitable for topical administration.

2. A topical composition comprising from about 0.25% w/w to about 3.0% w/w roflumilast, roflumilast N-oxide or pharmaceutically acceptable salt thereof, at least one additional active agent selected from about 0.001% w/w to about 0.5% w/w of tazarotene, from about 2% w/w to about 10% w/w benzoyl peroxide (BPO) and combinations thereof, and a carrier suitable for topical administration, wherein the carrier does not comprise hexylene glycol or diethylene glycol monoethyl ether.

3. The composition of claim 1, wherein all the active agents, comprising roflumilast, roflumilast N-oxide or pharmaceutically acceptable salt thereof, and at least one additional active agent selected from at least one retinoid and benzoyl peroxide, are encapsulated.

4. The composition of claim 2, wherein all the active agents, comprising roflumilast, roflumilast N-oxide or pharmaceutically acceptable salt thereof, tazarotene and benzoyl peroxide, are encapsulated.

5. The composition of claim 1, wherein at least one of the active agents, selected from roflumilast, roflumilast N-oxide or pharmaceutically acceptable salt thereof, at least one retinoid and benzoyl peroxide is encapsulated.

6. The composition of claim 2, wherein at least one of the active agents, selected from roflumilast, roflumilast N-oxide or pharmaceutically acceptable salt thereof, tazarotene and benzoyl peroxide is encapsulated

7. The composition of claim 1, wherein the active agents are fully dissolved or partly dissolved and partly suspended in a carrier suitable for topical administration comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof.

8. A dosage form comprising the composition of claim 1, wherein said composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe.

9. A method of treatment, prevention or alleviation of a skin disorder selected from acne and rosacea, said method comprises topical administration to the affected area of a subject with the skin disorder a therapeutically effective amount of a composition of claim 1.

10. The method of claim 9, wherein the skin disorder is acne, selected from acne vulgaris, papulopustular acne and nodular acne.

11. The method of claim 9, wherein the skin disorder is rosacea, selected from erythematotelangiectatic rosacea, papulopustular rosacea, rhinophyma rosacea and ocular rosacea.

12. The method of claim 9, wherein the treatment comprises once daily or twice daily topical administration to a subject in need thereof of a therapeutically effective amount of a composition comprising from about 0.25% w/w to about 3.0% w/w roflumilast, roflumilast N-oxide or pharmaceutically acceptable salt thereof at least one additional active agent selected from about 0.001% w/w to about 0.5% w/w of at least one retinoid, from about 2% w/w to about 10% w/w benzoyl peroxide (BPO) and combinations thereof, and a carrier suitable for topical administration.

13. The method of claim 9, wherein roflumilast, roflumilast N-oxide or pharmaceutically acceptable salt thereof and the at least one additional active agent exhibit an additive or synergistic effect, thereby allowing to reduce the amounts of the active agents in the composition.

14. A regimen of administration comprising the once daily or twice daily administration to an affected area of a subject with a skin disorder a therapeutically effective dose of the composition of claim 1 until the skin disorder is cured, prevented or alleviated.

15. A regimen of administration comprising the once daily or twice daily administration to a subject with a skin disorder a therapeutically effective amount of a dosage form of claim 8.

16. A kit comprising one or more dosage forms of claim 8 and instructions for use.
